(19) United States
(12) Patent Application Publication
(10) Pub. No.: US 2009/0081750 A1
(43) Pub. Date: Mar. 26, 2009

(54) TRANSFECTION IN MAGNETICALLY DRIVEN CONTINUOUS FLOW

(75) Inventors: Charles W. Ragsdale, Concord, CA (US); Teresa Rubio, El Cerrito, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA (US)

(21) Appl. No.: 12/023,706

(22) Filed: Jan. 31, 2008

Related U.S. Application Data
(60) Provisional application No. 60/889,869, filed on Feb. 14, 2007.

(51) Int. Cl. C12N 13/00; C12M 1/00

(52) U.S. Cl. 435/173.5; 435/285.1

(57) ABSTRACT

Biological cells and other membranous structures are transfected in a flow-through system by first rendering the structures magnetically active such that they respond to a magnetic field, suspending the structures in a solution of an exogenous species with which the structures are to be transfected, then placing the suspension in a channel and using a moving magnetization pattern along the channel wall to cause the structures to travel through the channel. Along their path of travel, the structures pass a transmitter that emits transfection energy sufficient to cause the exogenous species in the suspension to permeate the structure membranes and enter the interiors of the structures.

### CROSS-REFERENCE TO RELATED APPLICATION

This application claims the benefit of U.S. Provisional Patent Application No. 60/889,869, filed Feb. 14, 2007, the contents of which are incorporated herein by reference in their entirety.

### BACKGROUND OF THE INVENTION

1. Field of the Invention

This invention lies in the field of transfection of membranous structures such as biological cells, liposomes, and vesicles with species that are exogenous to the structures. In particular, this invention relates to the mobilization of the membranous structures to produce a continuous-flow transfection system.

2. Description of the Prior Art

Transfection is of value to research biologists and biochemists in the performance of various investigations and procedures, including siRNA experiments, research using cDNA libraries, and other clinical and research studies. Electroporation is one of the most advanced transfection technologies and involves the application of an electric field, typically in pulses, through a suspension of the membranous structures in a liquid solution of the exogenous species. It is believed that the electric field renders the membranes of the structures temporarily porous and thereby allows the species to penetrate the membrane. As the use of transfection has increased, certain concerns have arisen that have limited its applicability. One such concern is the low efficiency of the procedure and another is its variability. Both are believed to be attributable to the tendency of the membranous structures to aggregate and to the different orientations of the structures and the differences in exposure of the structures to the electric field when the structures are in different orientations. The low efficiency and the variability are also believed to be the result of a shielding effect of individual structures which limits the exposure of the shielded structures both to the electric field and to the exogenous species.

A further concern is the throughput rate, particularly in situations where the transfection is to be performed on large volumes of cells or other membranous structures. High-throughput systems have been developed for the simultaneous transfection of multitudes of samples differing in cell type, species to be injected, or both. Electroporation plates that accommodate large numbers of samples have been specifically designed for these transfections. Descriptions of such plates are found in International Patent Application Publication No. WO 2004/050866 A1, entitled “Large-Scale Electroporation Plates, Systems, and Methods of Use” (Genetronics, Inc., applicant; Gamelin, A., et al., inventors), published under the Patent Cooperation Treaty on Jun. 17, 2004; and in United States Patent Application Publication No. US 2007/0249036 A1, publication date Oct. 25, 2007, entitled “Apparatus for High-Throughput Electroporation” (inventors Ragsdale, C. W., et al.) and commonly owned herewith. High throughput has also been attempted by the simultaneous treatment of a large volume of membranous structures, more for example than can be accommodated in a single electroporation cuvette.

In the majority of the literature on electroporation and transfection, and in all of the commercially available electroporation systems, the procedure is performed in cuvettes in a batchwise format. With the high degree of manipulation and repetition involved in batchwise procedures, together with the size limitations of the typical cuvette, the processing of large volumes of sample and large numbers of structures is costly and prone to error. Continuous use of an electroporation chamber designed for batchwise use also entails a risk of overheating of the chamber which can result in irreparable rupture of the membranes of the cells being treated. Continuous-flow systems have been contemplated but have not been widely implemented. Descriptions of continuous-flow systems appear in Nicolau et al. (CBR Laboratories, Inc.), U.S. Pat. No. 5,612,207, “Method and Apparatus for Encapsulation of Biologically-Active Substances in Cells,” issue date Mar. 18, 1997, and Meserol, P. (EntreMed, Inc.), U.S. Pat. No. 6,090,617, “Flow Electroporation Chamber With Electrodes Having a Crystalline Metal Nitride Coating,” issue date Jul. 18, 2000. The electrodes in these patents are elongated strip electrodes, and electroporation is achieved by pumping the cells through the space between the electrodes, using a simple mechanical pump. The electroporation rate is limited by the pump rate, and there is little or no control over such factors as the density of the suspension at any particular point in the flow path and no way to eliminate differences in the exposure of individual cells to the electric field. Another description of a system with moving parts is found in Acker, J. L., et al., United States Patent Application Publication No. US 2004/0029240 A1, publication date Feb. 12, 2004. The system used by Acker et al. however involves moving electrodes and is not a flow-through system. The purpose of using moving electrodes is to impose a shear stress on the cells to continuously change the orientations of the cells.

Of further potential relevance to the background of the present invention is the use of electromagnetic radiation, such as pulses of light, to achieve transfection. In a manner analogous to electroporation, exposure of a membranous structure to a pulse of light energy can result in a transient permeabilization of the membrane without rupture of the membrane. As in electroporation, exposure to light energy is performed on cells suspended in a solution of a molecular species that is exogenous to the cells, thereby allowing the species to enter the cells through the permeated membrane. A description of this technique is found in Koller, M. R., et al. (Oncosis LLC), U.S. Pat. No. 6,753,161 B2, “Optoinjection Methods,” issue date Jun. 22, 2006. In this technique, the exposure to light and the resulting transient permeabilization are achieved while the cells are “substantially stationary.”

### SUMMARY OF THE INVENTION

The present invention resides in a system and method for the transfection of membranous structures in a continuous-flow format by utilizing magnetic forces to convey the structures through a channel and past a transmitter of transfection energy in the channel. The structures are made magnetically active either by injecting magnetic or magnetically responsive material into the structures or by adhering the structures to beads, particles, or partial enclosures that are either fabricated of magnetic or magnetically responsive material or contain such material. As in conventional transfection, the magnetically active structures are suspended in a solution of the exogenous species with which they are to be transfected, and the suspension is then drawn into the channel. Regions on a longitudinal wall within the channel that are arranged in a linear array are then magnetized in succession to create a moving magnetization pattern along the wall. The magnetization pattern draws the membranous structures or the beads, particles, or enclosures to which the structures are associated to the wall by magnetic attraction, and as the pattern moves along the wall, the structures move with the pattern. The magnetization pattern may include repelling forces as well as attracting forces to assist in the movement of the structures. The moving magnetization pattern causes the structures to travel through the channel in a controlled manner, preferably with controlled spacing between the structures and at a controlled rate of speed, and preferably while maintaining contact with the wall. During their course of travel, the membranous structures travel past a transfection energy transmitter. Upon reaching the energy field created by the transmitter, the structures will undergo transfection either one at a time or in groups of preselected size, at preselected time intervals and with a preselected spatial separation. In certain embodiments of the invention, the structures move in a single file along the wall and past the transfection energy transmitter, while in others, the structures move in clusters or groups, or in two or more parallel paths.

The moving magnetization pattern on the channel wall provides enough control over the movements of the membranous structures to allow pulses of the same intensity and duration to be applied to each structure. Uniform transfection of all, or substantially all, of the structures can thus be achieved at a high rate of efficiency with at most minimal loss from destruction of the structures due to exposure to excessive energy. Automated control over the magnetization of the surface regions on the wall also allows the system to accommodate membranous structures of different sizes and dimensions by selecting the number and spacing of the regions to be charged in the magnetization pattern. Automated control can also be used to vary the spacing between successive membranous structures, and to vary the number of structures that are exposed to the transmitter at any point in time.

As noted above, the moving magnetization pattern of this invention can be used to keep individual membranous structures separate from each other and to allow transfection to be performed uniformly on each structure as the structures pass in a single file through the transfection energy field. Preferably, the magnetic forces cause the membranous structures to establish physical contact with the wall of the channel and to maintain such contact as they travel through the channel. The structures are thereby prevented from aggregation, from clogging the channel, and from the shielding of one structure by another. Multiple structures can also be clustered on the surface of a magnetized (or magnetically responsive) bead to which the structures are bonded or on which they are grown. Successful and uniform transfection of all structures on a single supporting bead can be achieved by rotation of the bead while the bead is in the range of the transfection energy emitter. Once this is done, the magnetization pattern is shifted to cause the bead to leave the range of the emitter and be replaced by another bead. Rotation can be achieved by the incorporation of a flat piece of aluminum or other conductive but non-magnetic material in the bead, and imposing an alternating current (AC) across the bead. The current will set up eddy currents in the aluminum which will produce a repelling force between the aluminum and the AC field. This can provide rotation of the bead while also serving as an independent means of moving the bead in the desired direction of flow. If two or more membranous structures are adhered to the bead, the rotation can expose different structures in succession on the same bead to the transfection energy emitter, thereby assuring that all, or at least a high proportion, of the structures will undergo transfection. The system can also be used to obtain multiple exposures of a single structure to the transfection energy pulses.

These and other operations, functions, and advantages of this invention are explained in further detail below.

### BRIEF DESCRIPTION OF THE DRAWINGS

FIG. 1 is a perspective view of a transfection apparatus in accordance with the present invention.

FIG. 2 is a perspective view of the transfection apparatus of FIG. 1 with a portion of the channel wall removed to show the interior.

FIG. 3 is a perspective view of an alternative transfection apparatus, still in accordance with the invention.

### DETAILED DESCRIPTION OF THE INVENTION AND PREFERRED EMBODIMENTS

The membranous structures to which the present invention is applicable are bodies that are at least of macromolecular dimensions and include an enclosing membrane that under normal conditions is impenetrable to the species of interest. Examples of such membranous structures are liposomes, vesicles, organelles, and biological cells. Of biological cells, the invention applies to both prokaryotic and eukaryotic cells, and the cells can for example be animal cells, plant cells, yeast cells, human cells, or bacteria. Magnetism or magnetic responsiveness can be imparted to a membranous structure in ways known in the art. These include the injection of one or more magnetic particles or particles of magnetically responsive material into the membranous structure, and alternatively, the attachment of the membranous structure to a magnetic or magnetically responsive bead by surface functionalization of the structures for complexation with the bead, either through covalent binding, hydrophobic interaction, or affinity-type binding. The attachment of biological cells to magnetic or magnetically responsive beads can be achieved by growing the cells on the beads, using conventional cell growth techniques. Any of the membranous structures addressed by this invention can also be enclosed in cages or other partial enclosures of magnetic or magnetically responsive material, the enclosures securely grasping the structure while still offering access of the surrounding medium, including the exogenous species, to the structure.

The term “exogenous species” is used herein to denote any molecule or cluster of molecules that is not native to or otherwise present in the membranous structures, but also to denote molecules that are present inside the structure in only a limited quantity or at a limited concentration and of which an increase in the quantity or concentration is sought. Examples of classes of exogenous species that are typically inserted into membranous structures by transfection are nucleic acids, polypeptides, carbohydrates, lipids, and small molecules in general. Examples of nucleic acids are expression plasmids, expression cassettes and other expressible DNA, as well as RNA. The polypeptides can be antibodies, antibody fragments, enzymes, or proteins in general. The carbohydrates can be metabolites that are not naturally occurring, such as isotopically labeled sugars, labeled dextrans, and saccharides, oligosaccharides, and polysaccharides in general. Examples of small molecules are drugs, dyes, and ligands for endogenous receptors. Liposomes may serve as exogenous species when the membranous structures are bodies larger than liposomes.

The term “transfection energy” is used herein to denote any form of energy applied to a membranous structure that will render the membrane reversibly porous or otherwise permeable for a limited period of time sufficient to allow exogenous species in the suspending liquid to penetrate the membrane and enter the interior of the structure, and to do so without rupturing the membrane or otherwise causing irreparable damage to the structure. Examples of transfection energy are electrical energy (resulting in electroporation), light energy (both from a laser and from non-laser sources), thermal energy, RF energy, ultrasound, and electron beam energy. Preferred forms of transfection energy are electrical energy and laser light energy, applied either individually or in combination. Electrical energy (electroporation) is particularly preferred. The “transfection energy transmitter” is any device or component that will create a field of transfection energy, preferably one that is focused within a spatial volume of dimensions that are sufficiently small to limit the transfection to a preselected number of membranous structures at a time. The field can be small enough to accommodate only one structure at a time, or broad enough to accommodate a limited plurality such as two or more structures, or it can be a ray of energy sufficiently narrow to strike only one structure. Transmitters that are known in the art for each particular type of energy can be used. For electroporation, the transmitters can be electrodes; for light or thermal energy, the transmitters can be laser diodes. Other transmitters for these and other forms of transfection energy will be apparent to those skilled in the art.

The magnetizable regions on the longitudinal wall of the channel are fixed, stationary regions that can be individually and selectively magnetized by electromagnetic means. The regions are preferably conductors that are embedded in, or otherwise affixed to, the wall of the channel, in parallel strips or the exposed ends of poles, for example, with extensions that are wound with coils that can be individually energized to impart magnetism of either north or south polarity or neutrality. At a given point in time, the magnetization pattern will consist of regions or groups of regions with a given polarity alternating with regions or groups of opposite polarity or with non-magnetized regions or groups. With timed changes in polarity caused by changes in the current passing through the coils, the magnetization pattern will move through the regions and along the wall, producing a moving magnetic field in the channel that will carry the membranous structures with the moving field. Each region can have a separate, individual coil controlling the magnetism of the region, or the regions can be joined in groups of two or more regions with each group under the influence of a common coil separate from that of the other groups.

As noted above, the membranous structures can be rendered magnetically active by bombardment of the structures with particles of magnetically responsive material followed by use of a magnetic separation method to recover the structures that have been successfully impregnated with the particles. Particle bombardment techniques are known in the art and include, for example, the use of a pressurized stream of gas, explosive devices, or moving carrier plates to propel the particles. Alternatively, the structures can be coupled with particles or beads of magnetically responsive material without penetration, by surface functionalization and coupling, or by growing the structures on the surfaces of the particles or beads. Magnetically responsive metals and polymers can be used as the magnetically responsive material. Magnetically responsive particles, particularly polymers, can be functionalized for surface coupling to cells by the attachment of chemical moieties to the particle surface, the moieties being ones that will bond to or adhere to cells or to particular receptors on cells. Included among these moieties are functional groups for covalent binding or moieties that engage in affinity binding with the cell surface. Chemoattractants secreted by a healthy mammalian immune system can also be used as the functional groups, as can ligands that specifically bind to known cell surface receptors. The cells, particles, or both can also be coated with materials that favor attachment, such as charge groups or groups that promote hydrophobic binding. Other examples will be readily apparent to those skilled in the art.

The moving magnetization pattern on the wall preferably consists of two or more magnetizable regions that attract the membranous structures, or the beads or particles to which the structure are attached. The magnetization pattern can also consist of two or more magnetically attracting regions in combination with one or more magnetically repelling regions or magnetically neutral regions. When a region of repelling polarity is included, the repelling region may be positioned upstream (i.e., at the trailing end) of the region(s) bearing the attracting polarity to help propel the membranous structures forward in the direction of travel through the channel. The two polarities will thereby impose both a pushing force and a pulling force on each passing structure. Optimal control of the positions of the membranous structures during their travel through the channel can be achieved by a magnetization pattern containing two adjacent regions magnetized with an attracting polarity. When the spacing between these adjacent attracting regions is approximately equal to or slightly smaller than the length or diameter of the membranous structure, the two regions will stabilize the position of the structure. In certain embodiments, the system is adaptable by allowing the operator to select among different magnetization patterns to accommodate membranous structures of different sizes. Two magnetized regions can thus be separated by one or more intervening non-magnetized regions in the pattern, the number of intervening regions determining the spacing of the charged regions. In most cases, best results will be obtained with a center-to-center spacing of from about 0.1 micron to about 10 microns, and preferably from about 0.3 micron to about 3 microns, between magnetized or magnetizable regions.

The term “linear array” is used herein to indicate magnetizable regions that are arranged in a line, which can be either curved or straight, such that when the magnetization pattern is moved along the array the resulting magnetic field directs the membranous structures along a unidirectional path of travel. In most cases, a straight-line array will be most convenient. Two or more parallel linear arrays can be used as well, doubling or otherwise multiplying the capacity of the channel and the rate of transfection.

The term “magnetically active” is used herein to denote any material that is susceptible to the magnetized regions of the wall by being either attracted to or repelled by the regions when the regions are magnetized. Such materials include ferromagnetic materials, ferrimagnetic materials, paramagnetic materials, superparamagnetic materials, and diamagnetic materials. Iron, nickel, and cobalt are examples of ferromagnetic materials. Maghemite, magnetite, and ferrite are examples of ferrimagnetic materials. Aluminum, barium, and calcium are examples of paramagnetic materials. Further examples and examples of the other groups of magnetically active materials will be known to those skilled in the art.

The system can be designed to accommodate either a single cell or other membranous structure at a time passing through the channel or multiple structures. When the channel is long enough to accommodate two or more structures, the magnetization protocol will include a number of moving magnetization patterns equal to the number of structures. The spacing between adjacent magnetization patterns will preferably be sufficient to avoid interference between successive structures in their movement through the channel and in their exposure to the transfection energy from the transmitter. A spacing equal to ten or more structure diameters, and preferably fifty or more, will be appropriate in most cases.

The magnetization pattern can be designed to cause the membranous structures to travel in a single file, double file, or more. Travel in a single file is generally sufficient in most applications and can be achieved by limiting the dimensions of the magnetizable regions, the dimensions of the channel, or both. The travel velocity and number of structures passing through the channel per unit time can also vary. Preferably, the rate of travel is high enough to cause ten or more structures per second to pass the transfection energy transmitter, preferably 100 to 10,000 structures per second, and most preferably 300 to 3,000 structures per second.

The dimensions of the channel will nevertheless be large enough to allow the structures to flow freely through the channel without clogging the channel. A channel width or diameter of at least about 10 microns, preferably about 20 microns or greater, will be suitable in most cases, particularly for biological cells. Channels that are 1 mm of greater can also be used.

The transfection energy transmitter is positioned at a fixed location in the channel so that membranous structures during the course of their travel through the channel will come within the range of the transmitter. The transmitter can be a pair of electrodes to cause transfection by electroporation, and the electrodes can be regions on the surface that also serve as magnetizable regions. When the membranous structures approach these regions for transfection, these regions will be electrically charged rather than magnetized, and thereby serve as electrodes. The electrodes can thus be a pair of magnetizable regions, either on the same side of the channel or on opposing sides. The voltage applied between the regions for electroporation can vary, depending on the type of membranous structure, the dimensions of the channel and the spacing of the electrodes. In most cases, the voltage will be within the range of 0.3-30 V, preferably 1-5 volts.

Alternatives to electroporation are temperature-induced poration and light-induced poration. Either one can be achieved by the use of laser diodes or other transmitters of light energy. These transmitters can likewise be placed on one side of the channel or on opposing sides, and most effectively between an adjacent pair of magnetizable surface regions. Laser diodes will require little or no optics in view of their close proximity to the membranous structures.

For embodiments of the invention in which the membranous structures are adhered to beads that contain metallic strips and the channel wall features include the capability of forming an AC current to cause eddy diffusion in the metallic strips, the AC current can be imposed by electrifying the magnetizable regions. A suitably programmed controller can time the AC current and the magnetization to coordinate the linear movement of the membranous structures through the channel and the rotation of the structures once they come within the range of the transfection emitter.

While the features defining this invention are capable of implementation in a variety of constructions, the invention as a whole will be best understood by a detailed examination of a specific embodiment. One such embodiment is shown in the attached Figures.

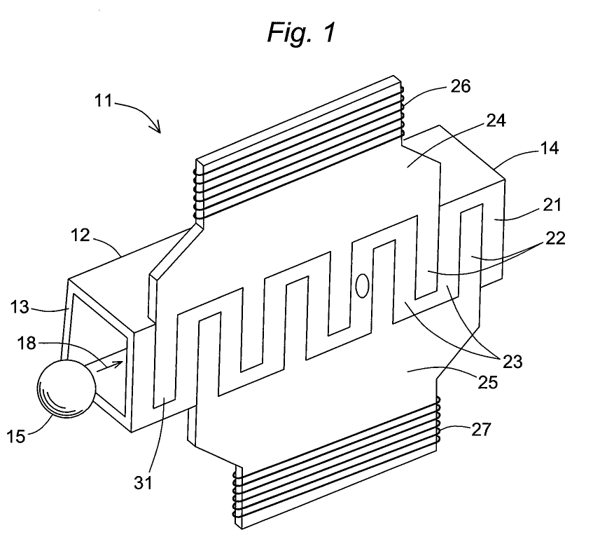

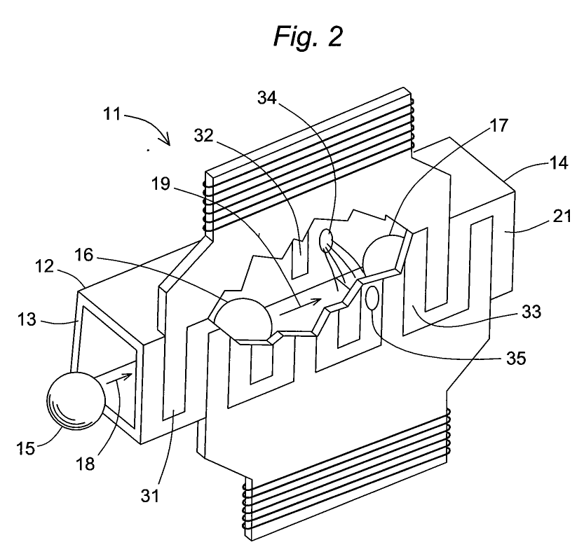

FIG. 1 depicts a continuous-flow transfection apparatus 11, and FIG. 2 depicts the same apparatus with a wall section removed to render the interior visible. The following description refers to both of these Figures.

The central part of the apparatus is a channel 12 that is open at both ends which, for convenience, are designated an entry end 13 and an exit end 14. A membranous structure 15 is shown entering the channel at the entry end, and in FIG. 2, further membranous structures 16, 17 are visible inside the channel. The structures move through the channel in the directions of the arrows 18, 19. One side wall 21 of the channel contains a series of magnetizable regions in the form of strips 22 of electrically conductive material that are magnetizable by electromagnetism. The strips are parallel and separated by gaps 23 of material that is electrically and magnetically insulating and therefore not magnetizable. The embodiment shown includes eight strips are arranged in two groups of four. The strips of each group are formed as tines of a fork, the tines of one group alternating with those of the other so that the linear array consists of alternating strips of the two groups. The four strips of each group are joined at a common juncture, the juncture 24 for the first group extending upward from the channel and the juncture 25 for the second extending downward. Coils 26, 27 of electrically conductive wire encircle each of the junctures, allowing the strips to be magnetized by passing an electric current through the coils.

In a typical procedure, the membranous structures are first rendered magnetically active by any of the methods described above and dispersed in a solution of the exogenous species that is to be inserted into the structures, prior to entry into the electroporation apparatus 11. As a magnetically active membranous structure 15 approaches the entry end 13 of the channel 12, an electric current is passed through the coil 26 that magnetizes the first group of strips, including the strip 31 nearest the entry end. As this strip 31 is magnetized, it attracts the membranous structure 15, drawing the structure into the channel and imparting a linear momentum to the structure in the direction of the arrows. Current to the coil 26 is then discontinued, and the coil 27 that magnetizes the second group of strips is energized, drawing the membranous structure to the second strip in the array. The energization of the coils 26, 27 in alternating manner is continued, drawing the structure 15 through the entire length of the channel. The moving magnetization pattern in this example is thus an alternating pattern. As an alternative to the construction shown, the channel 12 can contain two arrays of magnetizable strips, one on each side of the channel, forming a symmetrical moving magnetic field that will keep the membranous structures along the center line of the channel as they pass through.

A strip 32 on the opposing side wall of the channel and one of the strips 33 of the magnetizable strips can be connected to additional electric leads (not shown) to serve as electrodes for electroporation, and a voltage can be applied across these strips when the membranous structure passes between them. Alternatively, an independent pair of energy transmitters 34, 35 not associated with any of the magnetizable strips, can be used to achieved electroporation, or temperature-induced or light-induced poration. In embodiments involving structures that will respond to an AC current by producing an eddy current in the structure, the AC current can be imposed by any of the strips on the side wall of the channel.

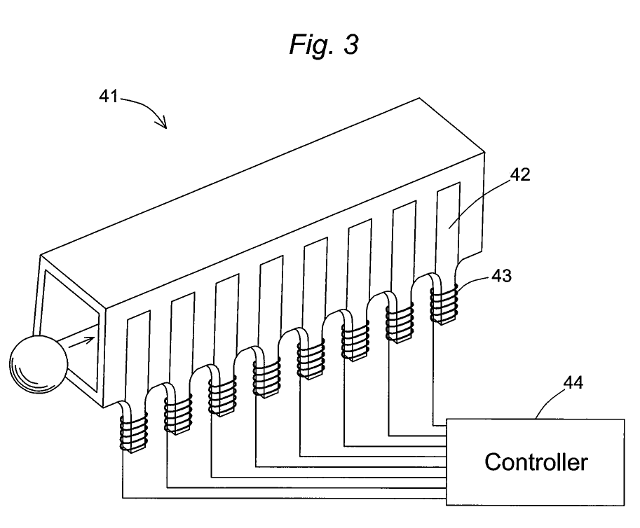

FIG. 3 depicts a variation 41 on the apparatus of FIGS. 1 and 2, in which each strip 42 is magnetized by an individual coil 43, and the energization of the coils is controlled by a programmable controller 44. The controller can thus be programmed to select the number of coils to be energized at any one time, the rate at which energization is performed and hence the speed of the magnetization pattern, and the strength of the magnetic field produced at each strip.

While the foregoing description describes various alternatives to the components shown in the Figures, still further alternatives will be apparent to those who are skilled in the art and are within the scope of the invention.

In the claims below, the terms “a” and “an” are intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of transfecting a plurality of magnetically active membranous structures with species exogenous to said structures, said method comprising:
(a) introducing a dispersion of said membranous structures in a liquid solution of said exogenous species into a channel to which is mounted a transfection energy transmitter, said channel comprising a longitudinal wall with a linear array of magnetizable surface regions;
(b) magnetizing said surface regions in succession to produce magnetic forces between said surface regions so magnetized and said membranous structures in a moving magnetization pattern that causes said membranous structures to travel along said longitudinal wall and past said transfection energy transmitter; and
(c) as each membranous structure passes said transfection energy transmitter, actuating said transfection energy transmitter to achieve said transfection.

2. The method of claim 1 wherein said moving magnetization pattern comprises surface regions that attract said membranous structures, alternating with surface regions that do not attract said membranous structures.

3. The method of claim 1 wherein said moving magnetization pattern comprises groups of two or more surface regions that attract said membranous structures alternating with groups of two or more surface regions that do not attract said membranous structures.

4. The method of claim 1 wherein said surface regions are sufficiently small to cause said membranous structures to travel past said transfection energy transmitter in a single file.

5. The method of claim 1 wherein said transfection energy transmitter is a pair of electroporation electrodes.

6. The method of claim 5 wherein said electroporation electrodes are positioned on opposing sides of said channel.

7. The method of claim 1 wherein said transfection energy transmitter is a laser diode.

8. The method of claim 1 wherein said step (b) comprises magnetizing said surface regions in succession at a rate causing said membranous structures to travel singly past said transfection energy transmitter at a rate of from 100 structures per second to 10,000 structures per second.

9. Apparatus for subjecting a plurality of magnetically active membranous structures in succession to transfection, said apparatus comprising:
a channel to which is mounted a transfection energy transmitter, said channel bounded by a longitudinal wall bearing a linear array of magnetizable surface regions;
transfection means for energizing said transfection energy transmitter to create an energy field sufficient to cause transfection of said membranous structures when said membranous structures are within said energy field; and
means for magnetizing said surface regions in succession to produce a moving magnetic field that attracts said membranous structures and thereby conveys said membranous structures in succession through said energy field.

10. The apparatus of claim 9 wherein said transfection energy transmitter is comprised of electroporation electrodes and said energy field is an electric field.

11. The apparatus of claim 9 wherein said transfection energy transmitter is comprised of a laser diode and said energy field is a light energy field.

12. The apparatus of claim 9 wherein said magnetizable surface regions are sufficiently small to cause said membranous structures to travel through said energy field in a single file.

13. The apparatus of claim 9 wherein said magnetizable surface regions have center-to-center spacings of from about 0.1 micron to about 10 microns.

14. The apparatus of claim 9 wherein said magnetizable surface regions have center-to-center spacings of from about 0.3 micron to about 3 microns.
